(19) United States
(12) Patent Application Publication
(10) Pub. No.: US 2026/0041810 A1
(43) Pub. Date: Feb. 12, 2026

(54) PHOTOCATALYST MODULE

(71) Applicant: Sharp Kabushiki Kaisha, Sakai City (JP)

(72) Inventor: Kazuya IWASE, Sakai City (JP)

(21) Appl. No.: 19/290,764

(22) Filed: Aug. 5, 2025

(30) Foreign Application Priority Data
Aug. 8, 2024 (JP) 2024-133328

(51) Int. Cl. A61L 9/20; B01D 53/00; B01D 53/86; B01D 53/88

(52) U.S. Cl. CPC A61L 9/205; B01D 53/007; B01D 53/8687; B01D 53/885; A61L 2209/12; B01D 2255/802; B01D 2257/90; B01D 2259/802

(57) ABSTRACT

The disclosure provides a photocatalyst module capable of reducing manufacturing costs, reducing size, reducing thickness, saving power, and reducing noise. A photocatalyst module according to the disclosure includes a housing including at least one air inlet and at least one air outlet, a light source unit disposed inside the housing, and a photocatalyst unit disposed inside the housing. The light source unit and the photocatalyst unit are provided so that the photocatalyst unit is irradiated with light from the light source unit. The light source unit includes a light source, and a heat dissipator provided to dissipate heat generated by the light source unit. The light source unit, the air inlet, and the air outlet are provided so that a flow of gas from the air inlet toward the air outlet is generated by the heat dissipated from the heat dissipator.

### CROSS-REFERENCE TO RELATED APPLICATION

The present application claims priority from Japanese Application JP2024-133328, the content to which is hereby incorporated by reference in to this publication.

### BACKGROUND ART

### Technical Field

The disclosure relates to a photocatalyst module.

### Background Art

A device is known that removes organic substances such as odorous substances in the air by utilizing photocatalytic activity. In such a device, for example, air is passed through a photocatalyst filter using a fan to remove the organic substances in the air.

### Summary

A known device is equipped with a fan for supplying air to a photocatalyst.

Therefore, a power supply unit for supplying power to a light source unit for irradiating the photocatalyst with light, and a power supply unit for driving the fan are required, and it is thus difficult to reduce the size and thickness of the device.

In addition, since noise is generated due to the driving of the fan, it is also necessary to consider the installation location.

The disclosure has been made in view of such circumstances, and provides a photocatalyst module capable of reducing manufacturing costs, reducing size, reducing thickness, saving power, and reducing noise.

The disclosure provides a photocatalyst module that includes a housing including at least one air inlet and at least one air outlet, a light source unit disposed inside the housing, and a photocatalyst unit disposed inside the housing. The light source unit and the photocatalyst unit are provided to cause the photocatalyst unit to be irradiated with light from the light source unit. The light source unit includes a light source, and a heat dissipator provided to dissipate heat generated by the light source unit. The light source unit, the air inlet, and the air outlet are provided to cause a flow of gas from the air inlet toward the air outlet to be generated by the heat dissipated from the heat dissipator.

According to the disclosure, a flow of gas from the air inlet toward the air outlet can be generated by the heat dissipated by the heat dissipator of the light source unit. Using this flow of gas, the gas can be supplied to the photocatalyst unit without a fan, and organic substances such as odorous substances contained in the gas can be removed. Therefore, the photocatalyst module according to the disclosure does not need to be equipped with a fan, and thus can reduce manufacturing costs, reduce size, reduce thickness, save power, and reduce noise.

### BRIEF DESCRIPTION OF DRAWINGS

FIG. 1 is a schematic cross-sectional view of a photocatalyst module according to an embodiment.

FIG. 2 is a schematic cross-sectional view of the photocatalyst module according to the embodiment.

FIG. 3 is a schematic cross-sectional view of the photocatalyst module according to the embodiment.

FIG. 4 is a schematic cross-sectional view of the photocatalyst module according to the embodiment.

### DESCRIPTION OF THE PREFERRED EMBODIMENTS

A photocatalyst module according to the disclosure includes a housing including at least one air inlet and at least one air outlet, a light source unit disposed inside the housing, and a photocatalyst unit disposed inside the housing. The light source unit and the photocatalyst unit are provided to cause the photocatalyst unit to be irradiated with light from the light source unit. The light source unit includes a light source, and a heat dissipator provided to dissipate heat generated by the light source unit. The light source unit, the air inlet, and the air outlet are provided to cause a flow of gas from the air inlet toward the air outlet to be generated by the heat dissipated from the heat dissipator.

It is preferable that the air inlet is disposed in a lower portion of the housing, the air outlet is disposed in an upper portion of the housing, and the light source unit is disposed in the lower portion of the housing.

The photocatalyst unit preferably includes a carrier sheet, and a photocatalyst supported or fixed on the carrier sheet.

The photocatalyst unit is preferably disposed to cause the gas flowing from the air inlet toward the air outlet to flow along the photocatalyst unit.

The photocatalyst unit is preferably provided to form a flow path through which the gas flowing from the air inlet toward the air outlet flows, and the flow path is preferably a flow path including a wide lower portion and a narrow upper portion.

The photocatalyst module according to the disclosure preferably includes a light guide.

The light source unit, the light guide, and the photocatalyst unit are preferably provided to cause the photocatalyst unit to be irradiated with the light from the light source unit through the light guide.

When the light guide is a light guide plate, the light guide plate is preferably disposed facing the photocatalyst unit, and the light source unit, the air inlet, and the air outlet are preferably provided to cause a flow of gas to be generated between the light guide plate and the photocatalyst unit.

When the light guide is a light guide rod, the photocatalyst unit is preferably disposed around the light guide rod, and the light source unit, the air inlet, and the air outlet are preferably provided to cause a flow of gas to be generated between the light guide rod and the photocatalyst unit.

It is preferable that the air inlet includes a first air inlet and a second air inlet, the first air inlet is closable by a first lid, and the second air inlet is closable by a second lid.

It is preferable that the air outlet includes a first air outlet and a second air outlet, the first air outlet is closable by a third lid, and the second air outlet is closable by a fourth lid.

An embodiment of the disclosure will be described below with reference to the drawings. Configurations illustrated in the drawings and the following description are examples, and the scope of the disclosure is not limited to the configurations illustrated in the drawings or the following description.

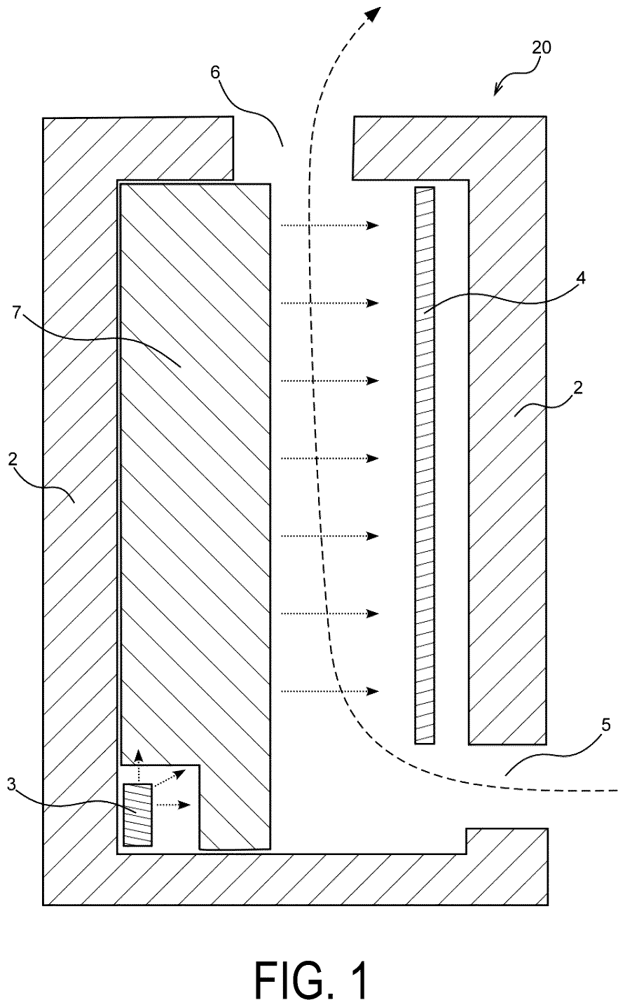

FIGS. 1 to 4 are each a schematic cross-sectional view of a photocatalyst module according to the present embodiment.

A photocatalyst module 20 according to the present embodiment includes a housing 2 including at least one air inlet 5, 5a, 5b, and at least one air outlet 6, 6a, 6b, 6c, a light source unit 3 disposed inside the housing 2, and a photocatalyst unit 4 disposed inside the housing 2. The light source unit 3 and the photocatalyst unit 4 are provided so that the photocatalyst unit 4 is irradiated with light from the light source unit 3. The light source unit 3 includes a light source, and a heat dissipator provided so as to dissipate heat generated by the light-source. The light source unit 3, the air inlet 5, 5a, 5b, and the air outlet 6, 6a, 6b, 6c are provided so that a flow of gas from the air inlet 5, 5a, 5b to the air outlet 6, 6a, 6b, 6c is generated by the heat dissipated from the heat dissipator.

The photocatalyst module 20 is a device that removes organic substances such as odorous substances in gas by using photocatalytic activity. The photocatalyst module 20 may be an air purification device, a deodorization device, or a sterilization device.

The housing 2 accommodates the light source unit 3 and the photocatalyst unit 4.

The housing 2 may also accommodate a light guide 7. Further, the housing 2 includes the air inlet 5, 5a, 5b and the air outlet 6, 6a, 6b, 6c.

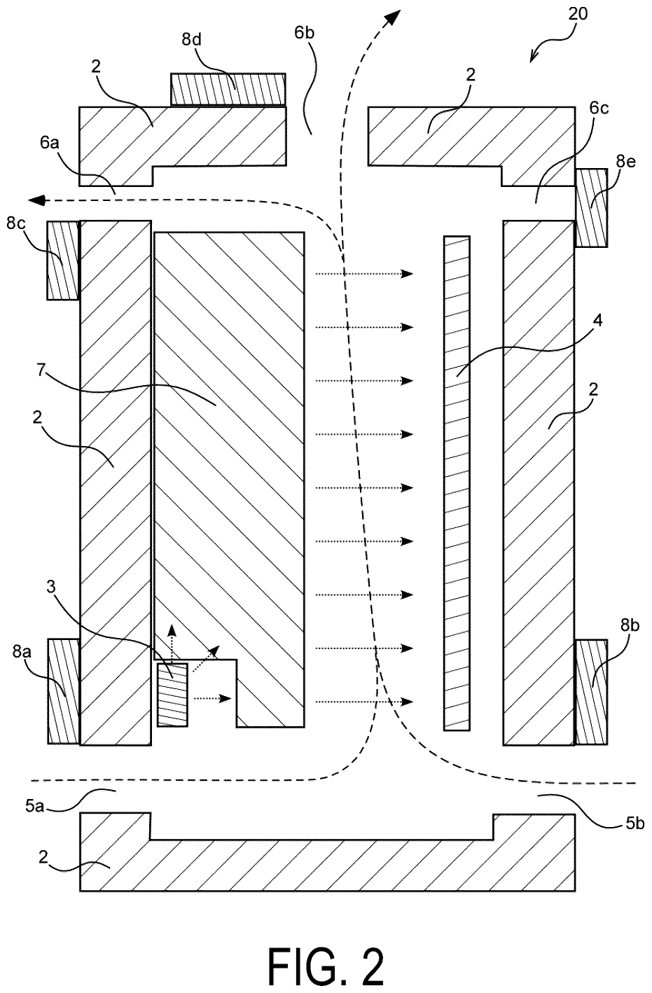

The air inlets 5, 5a, 5b are holes through which gas (for example, air) flows from the outside of the housing 2 into the inside of the housing 2. The housing 2 may include the single air inlet 5 or may include a plurality of air inlets, namely, the air inlets 5a, 5b. For example, the housing 2 included in the photocatalyst module 20 illustrated in FIG. 1 includes the single air inlet 5, and the housing 2 included in the photocatalyst module 20 illustrated in FIGS. 2, 3, and 4 includes the two air inlets 5a, 5b.

The air outlets 6, 6a, 6b, 6c are holes through which gas (for example, air) is discharged from the inside of the housing 2 to the outside of the housing 2. The housing 2 may have the single air outlet 6 or may have a plurality of air outlets, namely, the air outlets 6a, 6b, 6c. For example, the housing 2 included in the photocatalyst module 20 illustrated in FIGS. 1, 3, and 4 includes the single air outlet 6, and the housing 2 included in the photocatalyst module 20 illustrated in FIG. 2 includes the three air outlets 6a, 6b, 6c.

As in the photocatalyst module 20 illustrated in FIG. 2, the housing 2 may include the plurality of air inlets 5a, 5b, and the air inlets 5a, 5b may be provided so as to be closed by lids 8a, 8b. The housing 2 may include the plurality of air outlets 6a, 6b, 6c, and the air outlets 6a, 6b, 6c may be provided so as to be closed by lids 8e, 8d, 8e. In this way, some of the plurality of air inlets 5a, 5b or some of the plurality of air outlets 6a, 6b, 6c can be closed by the lid, the air flow inside the housing 2 can be changed. In addition, the positions of the air inlets 5a, 5b and the air outlets 6a, 6b, 6c can be changed according to a space in which the photocatalyst module 20 is installed, and organic substances such as odorous substances present in this space can be efficiently removed by the photocatalytic activity of the photocatalyst unit 4.

The light source unit 3 is a portion including the light source, and is disposed inside the housing 2. The light source unit 3 may be a light source device. The light source unit 3 is, for example, an LED lamp, a fluorescent lamp, an incandescent lamp, a halogen lamp, a xenon lamp, or the like. The light source unit 3 includes the light source (for example, a light-emitting diode), and the heat dissipator provided so as to dissipate the heat generated by the light source. The heat from the light source unit 3 may be dissipated from a portion other than the heat dissipator.

The light source unit 3 and the photocatalyst unit 4 are provided so that the photocatalyst unit 4 is irradiated with the light from the light source unit 3. In this way, organic substances such as odorous substances in the gas can be removed by the photocatalytic activity of the photocatalyst unit 4. The light emitted by the light source unit 3 may be irradiated to the photocatalyst unit 4 through the light guide 7. For example, the light source unit 3 and the photocatalyst unit 4 can be disposed as in the photocatalyst module 20 illustrated in FIGS. 1 to 4. In FIGS. 1 to 4, the direction of the light emitted by the light source unit 3 is indicated by dotted line arrows. In addition, by irradiating the photocatalyst unit 4 with the light emitted from the light source unit 3 through the light guide 7, the photocatalyst unit 4 disposed in a middle portion or an upper portion inside the housing 2 can be efficiently irradiated with the light emitted from the light source unit 3 disposed in a lower portion inside the housing 2.

The light guide 7 is a portion that guides light so that the photocatalyst unit 4 is irradiated with the light emitted from the light source unit 3. The light guide 7 can be disposed inside the housing 2. The material of the light guide 7 is, for example, acrylic resin, polycarbonate, glass, polystyrene, or the like. The shape of the light guide 7 may be a plate shape (light guide plate) or a rod shape (light guide rod).

The light guide 7 may have an uneven pattern that reflects or scatters light propagating inside the light guide 7. In this way, the light propagating inside the light guide 7 can be emitted to the outside through the uneven pattern. The light emitted from the light guide 7 is irradiated to the photocatalyst unit 4.

The light source unit 3 and the light guide 7 can be provided so that the light emitted from the light source unit 3 enters the light guide 7. In this way, the light emitted from the light source unit 3 can be caused to propagate inside the light guide 7.

When the light guide 7 is a light guide plate, the light guide plate is preferably disposed so as to face the photocatalyst unit 4. In this way, the entire photocatalyst unit 4 can be irradiated with the light emitted from the light source unit 3 through the light guide plate, and it is possible to cause the photocatalyst activity to be generated in the entire photocatalyst unit 4. In addition, the probability that the gas flowing from the air inlet 5, 5a, 5b to the air outlet 6, 6a, 6b, 6c comes into contact with the photocatalyst unit 4 can be increased, and organic substances such as odorous substances in the gas can be efficiently removed by the photocatalytic activity. The light guide 7 (light guide plate) can be disposed, for example, as in the photocatalyst module 20 illustrated in FIGS. 1 and 2.

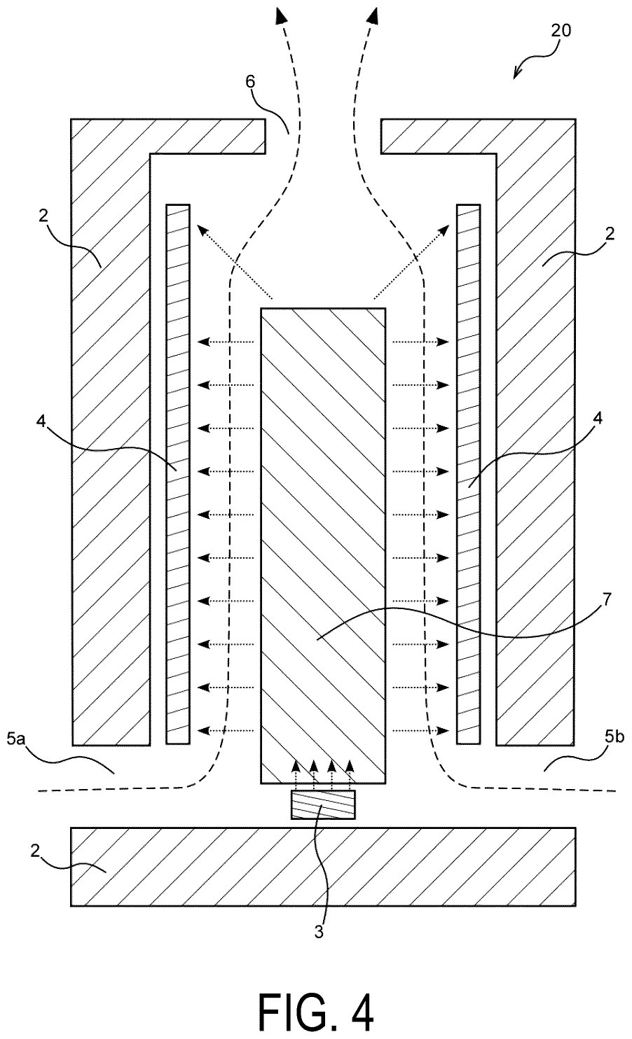

When the light guide 7 is a light guide rod, the photocatalyst unit 4 is preferably disposed around the light guide rod. In this way, the entire photocatalyst unit 4 can be irradiated with the light emitted from the light source unit 3 through the light guide rod, and it is possible to cause the photocatalyst activity to be generated in the entire photocatalyst unit 4. In addition, the probability that the gas flowing from the air inlet 5, 5a, 5b to the air outlet 6, 6a, 6b, 6c comes into contact with the photocatalyst unit 4 can be increased, and organic substances such as odorous substances in the gas can be efficiently removed by the photocatalytic activity. For example, the photocatalyst unit 4 may be formed in a cylindrical shape, and the light guide rod may be disposed inside the photocatalyst unit 4 having the cylindrical shape. The photocatalyst unit 4 can be disposed, for example, as in the photocatalyst module 20 illustrated in FIG. 4.

The light source unit 3, the air inlet 5, 5a, 5b, and the air outlet 6, 6a, 6b, 6c are provided so that a flow of gas (for example, air) from the air inlet 5, 5a, 5b toward the air outlet 6, 6a, 6b, 6c is generated by the heat dissipated from the heat dissipator of the light source unit 3. The temperature of the gas in the vicinity of the heat dissipator can be increased by the heat dissipated from the heat dissipator.

The gas with the increased temperature expands and the density of the gas decreases. Therefore, the gas becomes lighter and an upward air flow is generated inside the housing 2. The light source unit 3, the air inlet 5, 5a, 5b, and the air outlet 6, 6a, 6b, 6c can be disposed so that the flow of gas from the air inlet 5, 5a, 5b toward the air outlet 6, 6a, 6b, 6c is generated due to the upward air flow. In this way, the gas from the outside of the housing 2 can pass through the inside of the housing 2 without a fan. In addition, since the gas can be brought into contact with the photocatalyst unit 4 inside the housing 2, organic substances such as odorous substances contained in the gas passing through the inside of the housing 2 can be removed by the photocatalytic activity. Therefore, organic substances such as odorous substances present in the space in which the photocatalyst module 20 is installed can be removed.

For example, as in the photocatalyst module 20 illustrated in FIGS. 1 to 4, the air inlet 5, 5a, 5b can be disposed in a lower portion of the housing 2, the air outlet 6, 6a, 6b, 6c can be disposed in an upper portion of the housing 2, and the light source unit 3 can be disposed in the lower portion of the housing. In this way, the upward air flow can be generated inside the housing 2, and the flow of gas from the air inlet 5, 5a, 5b toward the air outlet 6, 6a, 6b, 6c can be generated (chimney effect). In FIGS. 1 to 4, the flow of gas is indicated by broken line arrows.

The photocatalyst unit 4 is a portion containing a photocatalyst (for example, photocatalytic particles). The photocatalyst is, for example, tungsten oxide, titanium oxide, or the like. The photocatalyst may contain catalytic promoter particles such as platinum group metal particles (for example, particles of Pt, Pd, Rh, Ru, Os, and Ir) on the surface thereof. By the photocatalyst receiving the light emitted from the light source unit 3, the photocatalytic activity can be generated, and organic substances such as odor substances in the gas flowing inside the housing 2 can be removed by the photocatalytic activity.

The photocatalyst unit 4 can include a carrier sheet, and a photocatalyst (for example, photocatalytic particles) supported or fixed on the carrier sheet. In this way, the probability that the gas flowing inside the housing 2 comes into contact with the photocatalyst can be increased, and organic substances such as odorous substances in the gas can be efficiently removed by the photocatalytic activity. The carrier sheet is, for example, paper, non-woven fabric, woven fabric, or the like. The light guide 7 and the photocatalyst unit 4 can be provided so that the photocatalyst unit 4 is irradiated with the light emitted from the light guide 7. In this way, the photocatalyst can be efficiently irradiated with the light, and the photocatalytic activity of the photocatalyst unit 4 can be increased.

The photocatalyst unit 4 is preferably disposed so that the gas flowing from the air inlet 5, 5a, 5b to the air outlet 6, 6a, 6b, 6c flows along the photocatalyst unit 4. In this way, the probability that the gas flowing inside the housing 2 comes into contact with the photocatalyst unit 4 can be increased, and organic substances such as odorous substances contained in the gas can be efficiently removed by the photocatalytic activity. In addition, it is possible to suppress an increase in resistance of the flow of gas from the air inlet 5, 5a, 5b toward the air outlet 6, 6a, 6b, 6c, and an air flow from the air inlet 5, 5a, 5b toward the air outlet 6, 6a, 6b, 6c can be generated by the heat dissipated from the heat dissipator. For example, the photocatalyst unit 4 can be disposed as in the photocatalyst module 20 illustrated in FIGS. 1 to 4.

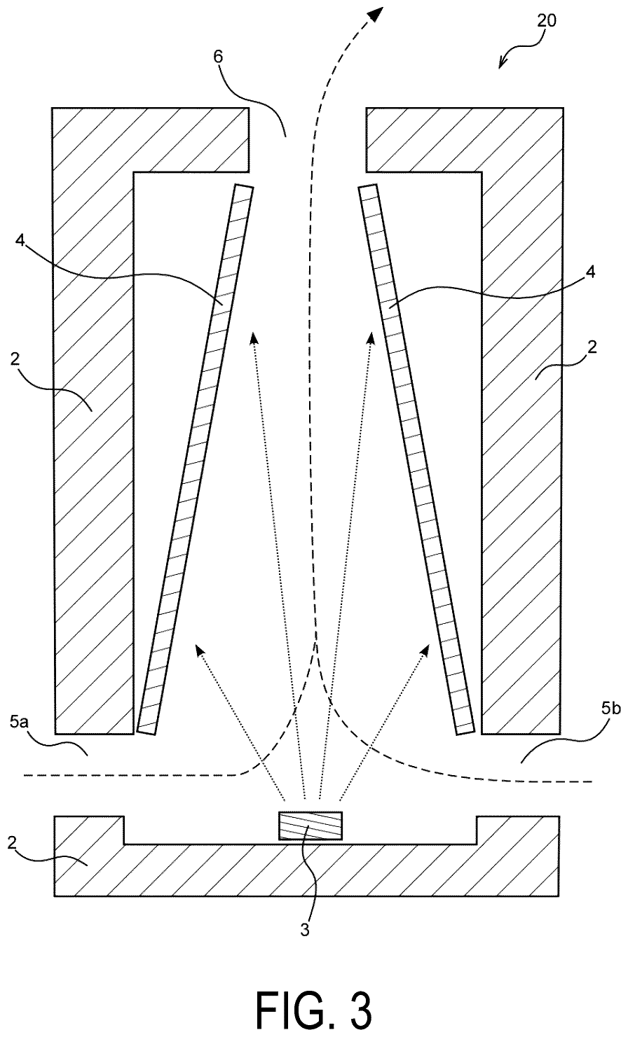

The photocatalyst unit 4 is preferably provided so as to form a flow path through which the gas flows from the air inlet 5, 5a, 5b to the air outlet 6, 6a, 6b, 6c, and the flow path is preferably a flow path including a wide lower portion and a narrow upper portion. In this way, the probability that the gas flowing inside the housing 2 comes into contact with the photocatalyst unit 4 can be increased, and organic substances such as odorous substances contained in the gas can be efficiently removed by the photocatalytic activity. In addition, an upward air flow is likely to be generated due to the heat dissipated from the heat dissipator of the light source unit 3, and the flow of gas from the air inlet 5, 5a, 5b to the air outlet 6, 6a, 6b, 6c is likely to be generated. For example, the photocatalyst unit 4 can be disposed as in the photocatalyst module 20 illustrated in FIG. 3.

When the light guide 7 is a light guide plate and the light guide plate is disposed so as to face the photocatalyst unit 4, the light source unit 3, the air inlet 5, 5a, 5b, and the air outlet 6, 6a, 6b, 6c are preferably provided so as to generate a flow of gas between the light guide plate and the photocatalyst unit 4. In this way, the probability that the gas flowing inside the housing 2 comes into contact with the photocatalyst unit 4 can be increased, and organic substances such as odorous substances contained in the gas can be efficiently removed by the photocatalytic activity. The light source unit 3, the air inlet 5, 5a, 5b, and the air outlet 6, 6a, 6b, 6c can be disposed, for example, as in the photocatalyst module 20 illustrated in FIGS. 1 and 2.

When the light guide 7 is a light guide rod and the photocatalyst unit 4 is disposed around the light guide rod, the light source unit 3, the air inlet 5, 5a, 5b, and the air outlet 6, 6a, 6b, 6c are preferably provided so that a flow of gas is generated between the light guide rod and the photocatalyst unit 4. In this way, the probability that the gas flowing inside the housing 2 comes into contact with the photocatalyst unit 4 can be increased, and organic substances such as odorous substances contained in the gas can be efficiently removed by the photocatalytic activity. The light source unit 3, the air inlet 5, 5a, 5b, and the air outlet 6, 6a, 6b, 6c can be disposed, for example, as in the photocatalyst module 20 illustrated in FIG. 4.

What is claimed is:

1. A photocatalyst module comprising:
a housing including at least one air inlet and at least one air outlet;
a light source unit disposed inside the housing; and
a photocatalyst unit disposed inside the housing,
wherein the light source unit and the photocatalyst unit are provided to cause the photocatalyst unit to be irradiated with light from the light source unit,
the light source unit includes a light source, and a heat dissipator provided to dissipate heat generated by the light source unit, and
the light source unit, the air inlet, and the air outlet are provided to cause a flow of gas from the air inlet toward the air outlet to be generated by the heat dissipated from the heat dissipator.

2. The photocatalyst module according to claim 1,
wherein the air inlet is disposed in a lower portion of the housing,
the air outlet is disposed in an upper portion of the housing, and
the light source unit is disposed in the lower portion of the housing.

3. The photocatalyst module according to claim 1,
wherein the photocatalyst unit includes a carrier sheet, and a photocatalyst supported or fixed on the carrier sheet.

4. The photocatalyst module according to claim 3,
wherein the photocatalyst unit is disposed to cause the gas flowing from the air inlet toward the air outlet to flow along the photocatalyst unit.

5. The photocatalyst module according to claim 3,
wherein the photocatalyst unit is provided to form a flow path through which the gas flowing from the air inlet toward the air outlet flows, and
the flow path is a flow path including a wide lower portion and a narrow upper portion.

6. The photocatalyst module according to claim 3, further comprising
a light guide,
wherein the light source unit, the light guide, and the photocatalyst unit are provided to cause the photocatalyst unit to be irradiated with the light from the light source unit through the light guide.

7. The photocatalyst module according to claim 6,
wherein the light guide is a light guide plate,
the light guide plate is disposed facing the photocatalyst unit, and
the light source unit, the air inlet, and the air outlet are provided to cause a flow of gas to be generated between the light guide plate and the photocatalyst unit.

8. The photocatalyst module according to claim 6,
wherein the light guide is a light guide rod,
the photocatalyst unit is disposed around the light guide rod, and
the light source unit, the air inlet, and the air outlet are provided to cause a flow of gas to be generated between the light guide rod and the photocatalyst unit.

9. The photocatalyst module according to claim 1,
wherein the air inlet includes a first air inlet and a second air inlet,
the first air inlet is closable by a first lid, and
the second air inlet is closable by a second lid.

10. The photocatalyst module according to claim 1,
wherein the air outlet includes a first air outlet and a second air outlet,
the first air outlet is closable by a third lid, and
the second air outlet is closable by a fourth lid.
